(19) United States
(12) Patent Application Publication
(10) Pub. No.: US 2023/0363687 A1
(43) Pub. Date: Nov. 16, 2023

(54) LULLABY ALGORITHM - FETAL PEAK DETECTION USING PERIODICITY

(71) Applicant: The Regents of the University of California, Oakland, CA (US)

(72) Inventors: Hung V. Cao, Irvine, CA (US); Tai N. Le, Irvine, CA (US); Michael P.H. Lau, Edmonds, WA (US); Tim Etchells, Seattle, WA (US); Daniel Jilani, Camarillo, CA (US)

(73) Assignee: The Regents of the University of California, Oakland, CA (US)

(21) Appl. No.: 18/198,200

(22) Filed: May 16, 2023

Related U.S. Application Data
(60) Provisional application No. 63/342,391, filed on May 16, 2022.

(51) Int. Cl. A61B 5/344; A61B 5/352; A61B 5/00

(52) U.S. Cl. CPC A61B 5/344; A61B 5/352; A61B 5/725

(57) ABSTRACT

A computer-implemented method for detecting fetal peaks in a maternal ECG signal includes steps of receiving the maternal ECG signal and preprocessing the maternal ECG signal wherein preprocessing includes removal of mECG R-peaks to form a process ECG signal that includes both fetal and noise peaks. Both fetal and noise peaks are detected using a peak detection algorithm which yields an array of peaks X_{k×1}. Permutations of all peak differences are calculated by subtracting elements of X_{k×1} from its transpose X_{k×1}^{T} to form a matrix M_{k×k}. Characteristically, elements in the matrix M_{k×k} corresponds to different sample periods that are used to determine peak positions. An array of periods denoted an array L_{P×1} is calculated. A 3-dimensional matrix G_{P×k×k} is constructed by dividing M_{k×k} by each element of L_{P×1}. An optimal M_{k×k} is determined by finding the M_{k×k} in the 3-dimensional matrix G_{P×k×k} that has it's first row with the most elements near a whole number.

### CROSS-REFERENCE TO RELATED APPLICATIONS

This application claims the benefit of U.S. provisional application Ser. No. 63/342,391 filed May 16, 2022, the disclosure of which is hereby incorporated in its(their) entirety by reference herein.

### TECHNICAL FIELD

In at least one aspect, the present invention relates to the detection of fetal peaks in a maternal ECG.

### BACKGROUND

Within the maternal ECG, fetal peaks and noise are often mixed together. The fetal and noise peaks must be separated from one another in order to have an accurate resolution of the fetal heartbeat in the fECG signal. The detection of fetal peaks in a maternal ECG for a real-time application must be quick, computationally light, accurate, and reliable for or while performing common everyday tasks.

Previous algorithm being tested for this problem was the k-means clustering algorithm. This algorithm determined the width, height, and height/width product as the features of the noise and fetal peaks and used clustering to separate them into noise and fetal peak clusters. Although these algorithms work reasonably well, clustered groups are ambiguous, another algorithm would be needed to determine which group is which. Finally, the implementation of K-means clustering in C-code is complex making its implementation in computationally light-weight systems.

Accordingly, there is a need for improved methods for extracting fECG peaks from mECG.

### SUMMARY

In at least one aspect, a system for detecting fetal peaks in a maternal ECG signal is provided. The system includes an ECG subsystem for collecting a maternal ECG signal that includes fetal peaks and a signal processing subsystem. The signal processing subsystem is configured to receives the maternal ECG signal and to preprocess the maternal ECG signal wherein preprocessing includes removal of mECG R-peaks to form a processed ECG signal that includes both fetal and noise peaks. The signal processing subsystem is further configured to detect positions of both fetal peaks and noise peaks using a peak detection algorithm and to create a plurality of sets of test peak positions wherein each set of test peak positions has its peak positions separated by an associated predetermined time period, the associated predetermined time period for each set being selected from a predetermined range of time period. Finally, the signal processing subsystem is further configured to align each set of test peak positions with detected positions of both fetal peaks and noise peaks to determine an optimal match with a subset of the detected positions of both fetal and noise peaks, the optimal match identify peaks corresponding to fetal peaks.

In another aspect, a computer-implemented method for detecting fetal peaks in a maternal ECG signal is provided. The method includes steps of receiving the maternal ECG signal and preprocessing the maternal ECG signal. Such preprocessing includes removal of mECG R-peaks to form a processed ECG signal that includes both fetal and noise peaks. Positions of both fetal peaks and noise peaks are detected using a peak detection algorithm. A plurality of sets of test peak positions is created. Characteristically, each set of test peak positions has its peak positions separated by an associated predetermined time period. The associated predetermined time period for each set are selected from a predetermined range of time periods. Each set of test peak positions are aligned with detected positions of both fetal peaks and noise peaks to determine an optimal match with a subset of the detected positions of both fetal peaks, the optimal match identify peaks corresponding to fetal peaks.

In another aspect, a computer-implemented method for detecting fetal peaks in a maternal ECG signal is provided. The method includes steps of receiving the maternal ECG signal and preprocessing the maternal ECG signal. The preprocessing includes removal of mECG R-peaks to form a processed ECG signal that includes both fetal and noise peaks. Both fetal and noise peaks are detected using a peak detection algorithm which yields an array of peaks Xₖ of dimension k′×1 where k′ is the number of peaks. Permutations of all peak differences are calculated by subtracting elements of Xₖ from its transpose Xₖ^{T} to form a matrix M_{i,j} of dimension k′×k′. Characteristically, elements in the matrix M_{k×k} corresponds to different sample periods that are used to determine peak positions. An array of periods denoted by vector L_{b} of dimension P is calculated where P is the number of time periods and b is a integer label having a value of 1 to P. A 3-dimensional matrix G_{b,i,j} is derived by dividing M_{i,j} by each element of L_{b}. An optimal M_{i,j} is determined by:
calculating permutations of all peak differences by subtracting elements of Xₖ from its transpose Xₖ^{T} to form a matrix M_{i,j} of dimension k′×k′, elements in the matrix M_{i,j} correspond to the different sample periods that are used to determine peak positions;
calculating an array of periods denoted by an array L_{b} of dimension P×1 where P is the number of time periods;
constructing a 3-dimensional matrix G_{b,i,j} by dividing M_{i,j} by each element of L_{b};
determining an optimal M_{i,j} by
   determining a temporal error for each element of G_{b,i,j} by rounding each element of G_{b,i,j} to the nearest whole number, subtracting G_{b,i,j} the rounded G_{b,i,j}, and then taking the absolute value of the result as indicated by:

Ĝ_{b,i,j}=|round(G_{b,i,j})−G_{b,i,j}|
summing the elements of Ĝ along its second dimension i to yield Z_{b,j} which represents the total temporal error of the i-th peak (with respect to column of Z) for each period (with respect to row of Z):
finding the row index r and column index c of Z_{b,j} with the minimum element will give an optimal period and an optimal reference peak respectively;
selecting the index of the 1st dimension as r and 3rd dimension as c of G_{b,i,j}, a weight vector is yielded such that Wᵢ=G_{r,i,c}; and
   Indexes with W less than a predetermined value are identified as the indexes corresponding to true fetal peaks fQRS in Xₖ.

In another aspect, the computer-implemented method a simple algorithm for fetal peak detection is provided.

In another aspect, the computer-implemented method allows positions of missing peaks to be estimated using a determined periodicity.

In another aspect, a computing-implemented method and wearable device for detecting fetal heartbeats are provided. Advantageously, detection can be performed directly on the patch rather than offloading to a cellular device or cloud service which might be unreliable (plus associated fees).

In another aspect, a Savitzky-Golay filter is applied for signal smoothing.

Advantageously, the computer-implemented method set forth herein is faster and less computationally intensive than prior art algorithms. Moreover, the computer-implemented method only uses simple matrix manipulations and peak detection to operate.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be made to the following detailed description, read in conjunction with the following drawings, wherein like reference numerals denote like elements and wherein:

FIG. 1A. Flowchart illustrating the Lullaby method for detecting fetal peaks in a maternal ECG signal using the periodicity of the fetal peaks as a feature.

FIG. 1B. Schematic of a system for detecting fetal peaks in a maternal ECG signal using the periodicity of the fetal peaks as a feature.

FIG. 2. Example of a suboptimal M_{k×k} matrix.

FIG. 3. Example of an optimized M_{k×k} matrix.

FIG. 4. Example fetal annotation and determination.

FIG. 5. Schematic of a computing system that implements the Lullaby method.

FIG. 6. Flow chart of Lullaby at a global level.

FIG. 7. An aECG segment with a set of detected peaks containing true and false fQRS. The true fQRS are extracted as the peaks that fit the period To=459 samples.

FIG. 8. Flowchart of the calibration phase of Lullaby.

FIG. 9. Flowchart of the real-time phase of Lullaby.

FIG. 10. Bland-Altman Analysis of average heart rate derived by the true fQRS and the average PTF.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to formula or elements thereof, lowercase subscripted letters are integer labels.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

When referring to a numerical quantity, in a refinement, the term “less than” includes a lower non-included limit that is 5 percent of the number indicated after “less than.” A lower non-includes limit means that the numerical quantity being described is greater than the value indicated as a lower non-included limited. For example, “less than 20” includes a lower non-included limit of 1 in a refinement. Therefore, this refinement of “less than 20” includes a range between 1 and 20. In another refinement, the term “less than” includes a lower non-included limit that is, in increasing order of preference, 20 percent, 10 percent, 5 percent, 1 percent, or 0 percent of the number indicated after “less than.”

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

The term “electrical signal” refers to the electrical output from an electronic device or the electrical input to an electronic device. The electrical signal is characterized by voltage and/or current. The electrical signal can be stationary with respect to time (e.g., a DC signal) or it can vary with respect to time.

It should be appreciated that in any figures for electronic devices, a series of electronic components connected by lines (e.g., wires) indicates that such electronic components are in electrical communication, optical communication, or wireless communication with each other. Moreover, when lines directed connect one electronic component to another, these electronic components can be connected to each other as defined above.

The processes, methods, or algorithms disclosed herein can be deliverable to/implemented by a processing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, organic storage such as DNA, and other magnetic and optical media. The processes, methods, or algorithms can also be implemented in a software executable object. Alternatively, the processes, methods, or algorithms can be embodied in whole or in part using suitable hardware components, such as Application Specific Integrated Circuits (ASICs), quantum processors, Field-Programmable Gate Arrays (FPGAs), state machines, controllers or other hardware components or devices, or a combination of hardware, software and firmware components.

The term “QRS” refers to a complex of waves seen on an electrocardiogram that represents the electrical activity that occurs during ventricular depolarization. The QRS complex is composed of three main waves: the Q wave, the R wave, and the S wave. The Q wave is the first downward deflection seen, the R wave is the first upward deflection after the Q wave, and the S wave is the second downward deflection following the R wave.

Throughout this application, where publications are referenced, the disclosures of these publications in their entireties are hereby incorporated by reference into this application to more fully describe the state of the art to which this invention pertains.

### Abbreviations

“ASIC” means application specific integrated circuit.
“aECG” means abdominal electrocardiogram.
“mECG” means maternal electrocardiogram.
“ECG” means electrocardiogram.
“fECG” means fetal electrocardiogram.
“fQRS” means fetal QRS.
“fHR” means fetal heart rate.
“mQRS” means maternal QRS.
“PTF” means Periodic Trend Feature.

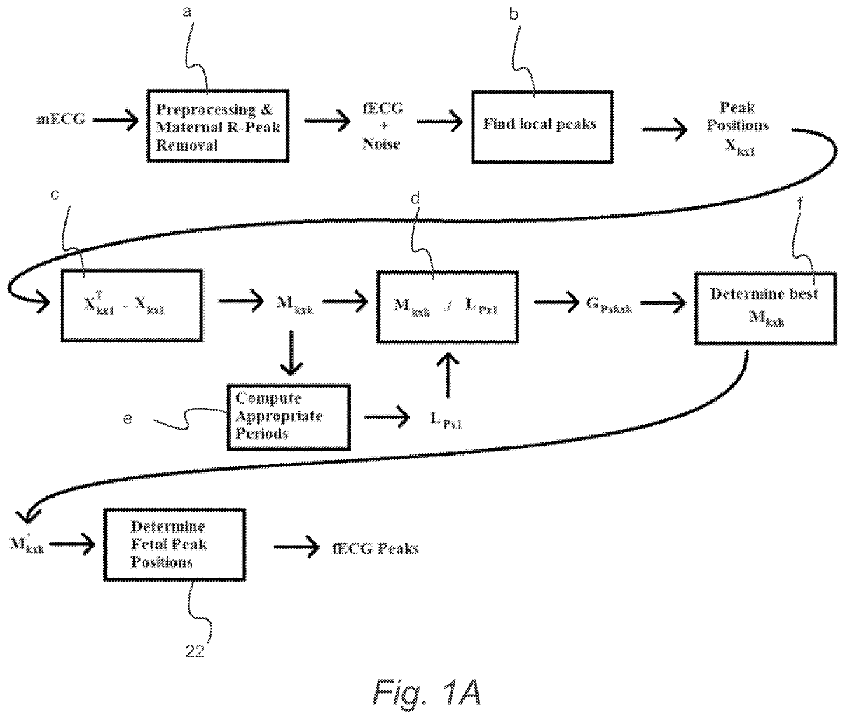

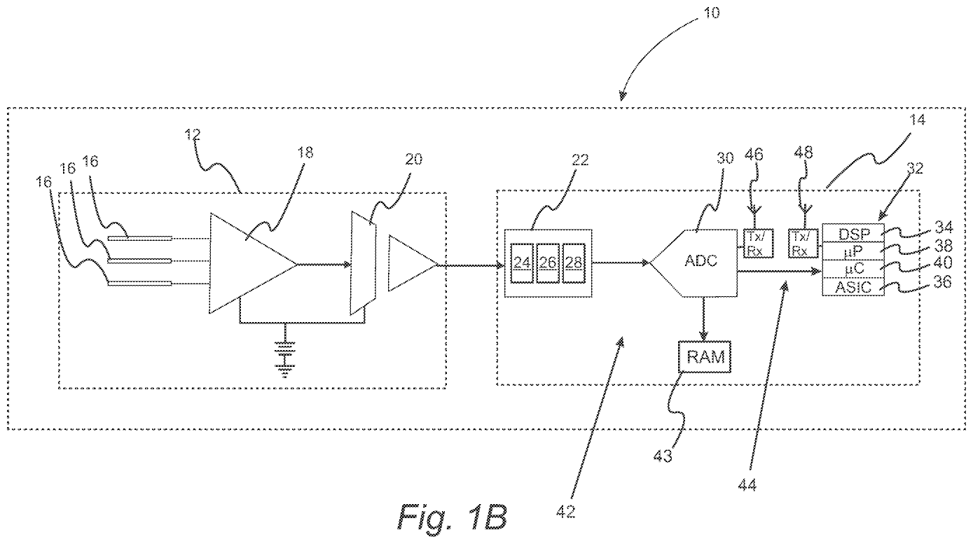

Referring to FIGS. 1A and 1B, schematics showing as system and method for detecting fetal peaks are provided. FIG. 1A provides a flowchart illustrating the Lullaby method for detecting fetal peaks in a maternal ECG signal using the periodicity of the fetal peaks as a feature. FIG. 1B provides a schematic of a system that performs the steps of FIG. 1A. System 10 includes an ECG subsystem 12 for collecting a maternal ECG signal that includes fetal peaks and a signal processing subsystem 14 for implementing the methods set forth herein. In a refinement, the maternal ECG signal is an abdominal ECG signal. In step a), a maternal ECG signal is received from ECG subsystem 12 and preprocessed by signal processing subsystem 14. The maternal ECG signal is generated from a plurality of electrodes 16. In general, there will be at least 3 electrodes. A typical ECG system can have from 3 to 10 electrodes. Moreover, a patch electrode system can have 3, 5 or 7 electrodes. ECG subsystem 12 includes amplification system 18 and isolation amplifier or buffer 20 in electrical communication with amplification system 18. The preprocessing step involves removing baseline wander, signal smoothing, and the removal of mECG R-Peaks. In this regard, signal processing subsystem 14 can include signal filtering subsystem 22 that can include one or more of highpass filder 24, low pass filter 26, and notch filter 28. In a refinement, baseline wander is removed using a high pass filter 22. In a variation, the maternal ECG signal is digitized prior to processing. In a refinement, the analag out output from the filtering subsystem is converted to a digital signal by analog-to-digital converter 30 for latter processing. The signal can be smoothed by using a Savitzky-Golay filter or another suitable smoothing algorithm. Such smoothing algorithms are deployed by digital processing subsystem 32. Moreover, signal filtering subsystem 22 or digital processing subsystem 32 can be configured to remove the mECG R-Peaks.

In a variation, the signal processing subsystem 14 includes a digital signal processor 34 configured to configured to prepocess the maternal ECG signal. In a variation, the signal processing busystem 14 includes application specific integrated circuits 36 configured to execute steps a) to e). In another variation, the signal processing system includes a computer processor 38 or microcontroller 40 configured to execute steps a) to e).

In a variation, ECG subsystem 12 includes 2 to 4 electrodes 16 for collecting the maternal ECG signal, circuitry 42 for storing (e.g., memory 43) and/or processing the maternal ECG signal, and an interface 44 for communicating with a computing device. Interface 44 can provide a wired connection to computing device 40 or a wireless connection (e.g, Bluetooth) using transmitters and receivers along with antennas 46, 48.

In another aspect, digital processing subsystem 32 can be configured to execute step b) in which, with the mECG R-Peaks removed, the local peaks in the ECG signal containing both fetal and noise peaks can be detected using a peak detection algorithm which yields a vector (i.e., a one-dimensional array) of peaks Xₖ of dimension k′ where k′ is the number of peaks and where k is the integer label of the array/vector running from 1 to k′.

Digital processing subsystem 32 is also configured to execute step c) in which permutations of all peak differences are obtained by subtracting the elements of Xₖ from its transpose Xₖ^{T} to form matrix M_{i,j} of dimension k′×k′ (i.e., a matrix with elements M_{i,j} with i and j independently being 1 to k′). Matrix M_{i,j} can be referred to as M_{k×k} for simplicity where i, j, and k are integer labels ranging from integer values of 1 to k′

The elements of M_{i,j} correspond to the different sample periods that can potentially be used to determine the peak positions. However, not all periods are appropriate for the certain range of periods that should be expected from a healthy fetal heartbeat. In a refinement, the array of periods includes each integer in a predetermined range of integers. For example, the predetermined range of integers is determined from a range of periods determined from 40 to 200 heart beats per minute. Therefore, digital processing subsystem 32 can be configured to execute step e) in which a vector of appropriate periods denoted by L_{b} of dimension P is computed where P is the number of time periods. The letter b is an integer label for the elements of the array running from 1 to P. In a refinement, the vector of appropriate periods is determined.

Digital processing subsystem 32 is also configured to execute step d) in which a 3-dimensional matrix G_{b,i,j} is constructed by dividing M_{i,j} by each element of L_{b} where b, i, and j are integer labels.

Digital processing subsystem 32 is also configured to execute step f) in which an optimal M_{i,j} is determined by determining a temporal error for each element of G_{b,i,j} by rounding each element of G_{b,i,j} to the nearest whole number, subtracting G_{b,i,j} the rounded G_{b,i,j}, and then taking the absolute value of the result as indicated by:

Ĝ_{b,i,j}=|round(G_{b,i,j})−G_{b,i,j}|.

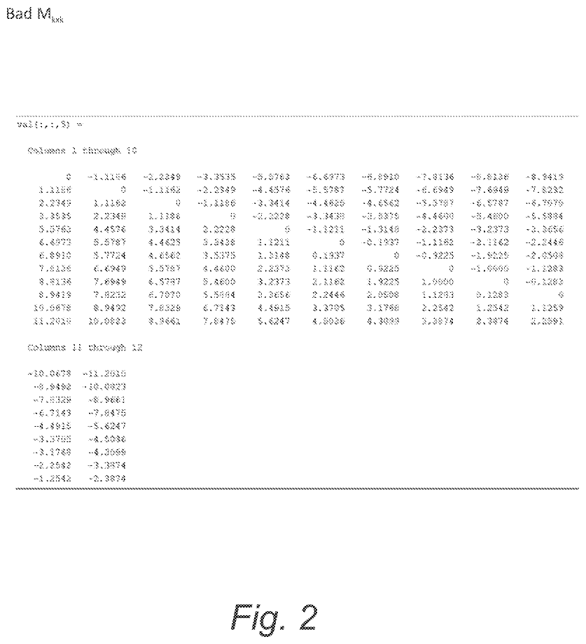

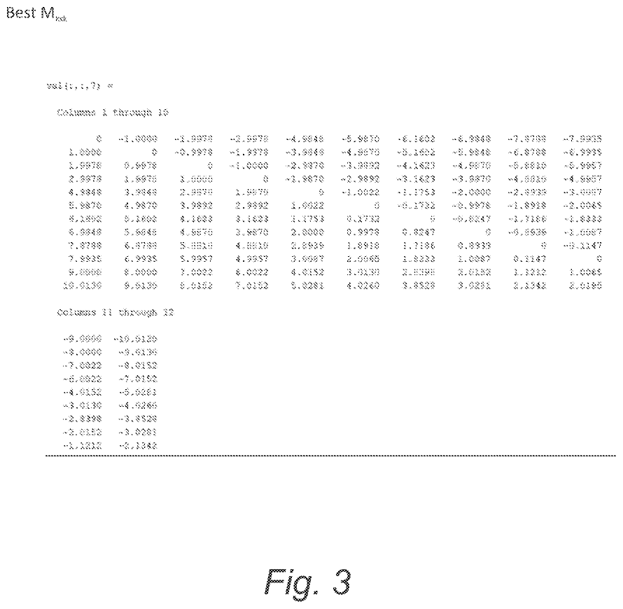

The elements of Ĝ are summed along its second dimension i to yield Z_{b,j} which represents the total temporal error of the i-th peak (with respect to column of Z) for each period (with respect to row of Z). Next, the method includes finding the row index r and column index c of Z_{b,j} with the minimum element will give an optimal period and an optimal reference peak respectively. The 1st dimension r and 3rd dimension c of G_{b,i,j} are selected as Wᵢ=G_{r,i,c} wherein r, c, i, j, and b are integer labels. FIG. 2 provides an example of a suboptimal M_{k×k} while FIG. 3 provides an example of an optimal M_{k×k}.

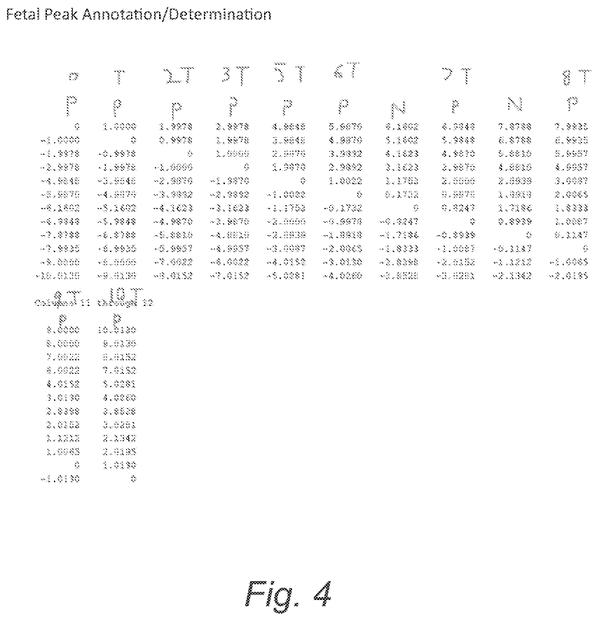

Digital processing subsystem 32 is also configured to execute step g) in which indexes with W less than a predetermined value are identified as corresponding to true fetal peaks fQRS in Xₖ. The predetermined value is typically less than 0.3. FIG. 4 provides an example of fetal annotation and determination of the fetal peaks.

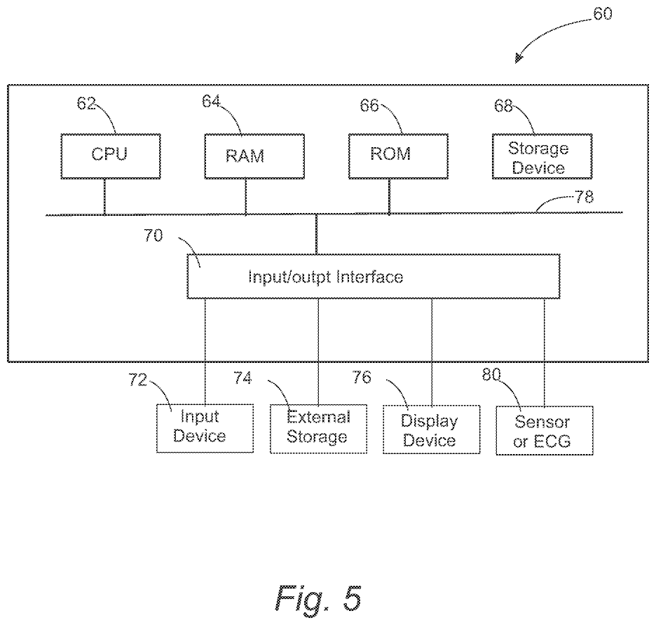

FIG. 5 provides a block diagram of a computing system that can be used to implement the methods. Each computing device of computing device 60 includes a processing unit 62 that executes the computer-readable instructions set forth herein. Processing unit 62 can include one or more central processing units (CPU) or micro-processing units (MPU). Computing device 60 also includes RAM 64 or ROM 66 having instructions for the Lullaby method encoded therein. Computing device 60 can also include a secondary storage device 68, such as a hard drive. Input/output interface 70 allows interaction of computing device 60 with an input device 72 such as a keyboard and mouse, external storage 74 (e.g., DVDs and CDROMs), and a display device 76 (e.g., a monitor). Input/output interface 70 allows interaction with sensor 80 for inputing the ECG data or a derivative thereof. Processing unit 62, the RAM 64, the ROM 66, the secondary storage device 68, and input/output interface 70 are in electrical communication with (e.g., connected to) bus 78. During operation, computing device 60 reads computer-executable instructions (e.g., one or more programs) recorded on a non-transitory computer-readable storage medium which can be secondary storage device 68 and or external storage 74. Processing unit 62 executes these reads computer-executable instructions for the computer-implemented methods set forth herein. Specific examples of non-transitory computer-readable storage medium for which executable instructions for computer-implemented methods are encoded onto include but are not limited to, a hard disk, RAM, ROM, an optical disk (e.g., compact disc, DVD), or Blu-ray Disc (BD)™), a flash memory device, a memory card, and the like.

Advantageously, the methods and system providing herein can be used to detect a number of medical conditions. For example, monitoring the fetal heart rate can indicate fetal distress or hypoxia which can be treated by administering oxygen to the mother. In some situations, intravenous fluids can be given to the mother to maintain hydration and support blood flow to the placenta. Fetal heart rate monitoring can be used to assess the well-being of the fetus during preterm labor. In this situation, tocolytic medications may be administered to the mother to suppress uterine contractions. Alternatively, emergency delivery may be performed is the fetal heart rate pattern indicated serve distress. Fetal heart rate monitoring can provide insights into the fetal growth and development warranting close monitoring of fetus and mother.

The following examples illustrate the various embodiments of the present invention. Those skilled in the art will recognize many variations that are within the spirit of the present invention and scope of the claims.

I. Materials & Methodology

A. Data Set

Lullaby is evaluated on the Physionet 2013 challenge dataset [1] set A which contains 75 1-minute abdominal ECG recordings sampled at 1000 Hz (fs). The data also includes annotations of the fECG taken directly from the fetus using fetal scalp electrodes.

B. Process Flow

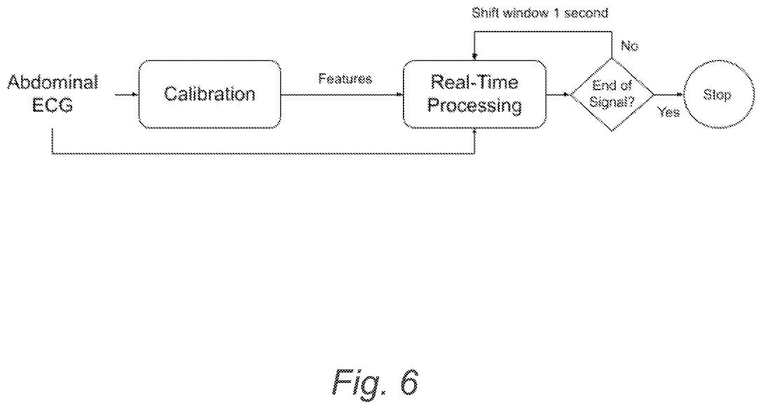

Lullaby operates in two phases called the ‘Calibration’ and ‘Real-Time’ phases. The calibration phase calculates the prominence and width of the fQRS. The prominence and width of the fQRS calculated in the calibration phase are used as features for fQRS detection in the real-time phase. The real-time phase simulates the extraction of the fQRS in real-time with a 1-second delay from the recording time. These are illustrated in FIG. 6.

C. Preprocessing

The aECG (Xₐ) can be considered a linear combination of the fECG (X_{f}), mECG (Xₘ), and noise (Xₙ):

Xₐ=X_{f}+Xₘ+Xₙ (1)

To maximize the effectiveness of the fQRS extraction, the aECG's baseline wander (BW) and maternal QRS complexes (mQRS) are removed from the signal. BW is a low frequency noise component that causes the aECG to oscillate along a low frequency sinusoidal wave. To model BW, a 100-point moving average filter is applied to the aECG and then subtracted from the aECG:

$$Y [ n ] = {X}_{a} [ n ] - \frac{1}{N} {\sum }_{k = 1}^{N} {X}_{a} [ n - N + k ] , N \leq 100 \qquad ( 2 )$$

where Y represents the aECG with the baseline removed, and N is the number of observable samples.

The mQRS are usually the most prominent morphological feature in the aECG signal, and thereby need to be removed in order to reliably detect the less prominent fQRS. This is done by first re-orienting the mQRS to face upwards:

$$\hat{Y} = \{ \begin{matrix}Y , & if \vert min ( Y ) \vert \leq max ( Y ) \\ - Y , & if \vert min ( Y ) \vert > max ( Y )\end{matrix} \qquad ( 3 )$$

A peak finding algorithm is applied which detects the K most prominent peaks in Y\hat{ }, where K is the duration in seconds of the ECG segment being processed. A secondary peak finding algorithm is applied which detects peaks at a height higher than 75% of the median height of the first set of peaks. This second set of peaks are mostly the mQRS within aECG. The duration of the mQRS complex is on average 100 ms, therefore all indexes within 50 ms (50 samples) of the mQRS are set to zero. In total these prepossessing steps reduces the aECG signal down to its fECG component.

D. Periodic Trend Feature

The fECG can be approximately modeled as a periodic signal with the fQRS being the repetitive morphology. A period To can be used to approximate the separation of the fQRS, and likewise be used to distinguish between true and false fQRS. Consider a B-length vector ipB which contains a mix of true and false fQRS indexes in ascending order. By determining the permutation of all delays between the fQRS in Ψ_{B} with respect to To, the indexes of true fQRS in Ψ_{B} which best follow the periodic trend To are distinguishable:

$${Ψ}_{B , B} = \frac{{ψ}_{B} - {ψ}_{B}^{T}}{{T}_{o}} \qquad ( 4 )$$

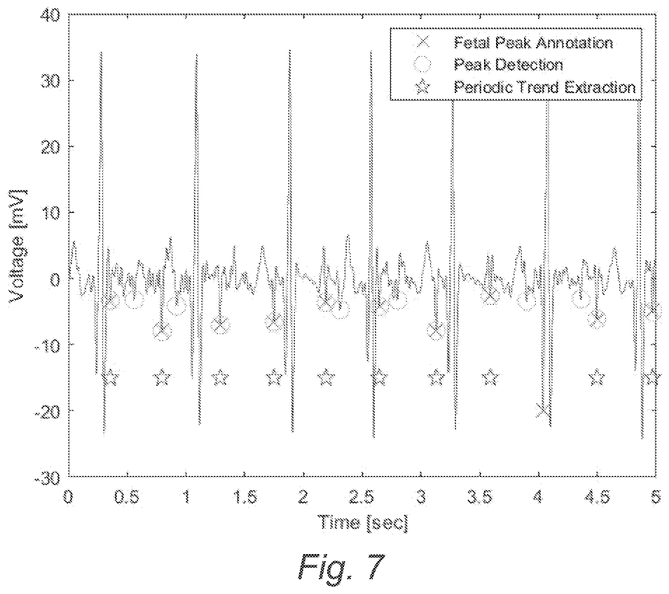

The row and column of elements of Ψ_{B,B} within 0.15 of an integer value can be used to identify the indexes of Ψ_{B} which correspond to the fQRS as shown in FIG. 7.

To determine the best value of To to approximate the fQRS separation, Lullaby tries all integer To values. The fHR can be between 80 and 180 bpm. This corresponds to a range of fQRS intervals between 333 ms (333 sample) to 750 ms (750 sample) which can be expressed as a set L:

L= [symbol] 333 334 . . . 749 750 [symbol] (5)

The rows and columns of Ψ_{B,B} lie along the i-th and j-th dimensions respectively. However if Ψ_{B,B} was divided by L along a b-dimension perpendicular to the ij-dimensions, all possible ΨΨ_{B,B} would be computed in a single 3-D matrix G:

$${G}_{b , i , j} = \frac{{ψ}_{B} - {ψ}_{B}^{T}}{{L}_{k}} \qquad ( 6 )$$

To determine which index of Ĝ would yield the best ΨΨ_{B,B}, the difference between the elements of Ĝ to the nearest integer must be computed and summed along the i-th dimension:

G=|└G┐−G| (7)

Z_{b,j}=Σᵢ₌₁^{B}Ĝ_{b,i,j} (8)

Elements of Z represent the total deviation of the fQRS from the periodic trend To grouped along the i-th dimension. The row (r) and column (c) indexes of the smallest group deviation of Z thereby correspond to the indexes of the best value of To and best ΨB,B column to evaluate along respectively:

$$r , c = arg {min}_{k , j} {Z}_{k , j} \qquad ( 9 )$$

$${W}_{i} = {G}_{r , i , c} \qquad ( 10 )$$

The indexes where W is less than 0.15 correspond to the true fQRS indexes in ΨB.

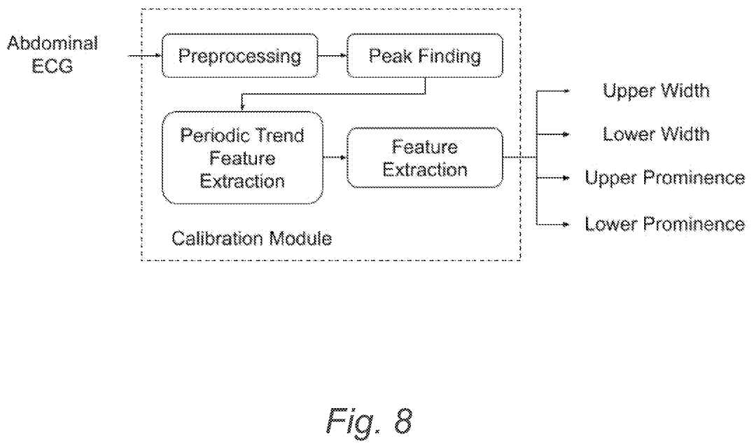

The ‘calibration’ phase (FIG. 8) of Lullaby determines the range of desired widths and prominence that correspond to the fQRS. The process begins by prepossessing the first 12-seconds of the aECG. Peak finding is applied to detect a set of peaks and valley with widths between 5 and fₛ/35. This set is then trimmed down by only keeping peaks and valleys with prominence greater than ⅓ of the most prominent peak or valley. Peaks and valleys are finally required to be at least 50 samples apart. The periodic trend feature (PTF) is applied to the remaining peaks to remove the false fQRS. From these calibrated peaks, the upper and bottom bounds of the width feature for the desired fQRS are computed as 2 standard deviations above and below the median width respectively. The same calculations are applied for the prominence.

F. Real-Time

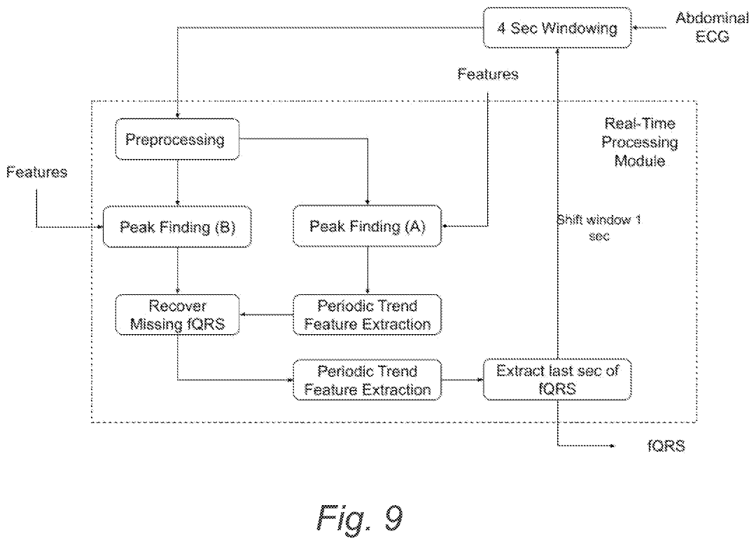

The ‘real-time’ phase (FIG. 9) of Lullaby extracts the fQRS from the most recent 1 second of data, in-order to simulate real-time extraction. During the real-time phase, the aECG is processed in 4-second batches with the aECG being shifted 1 second in time after each completed cycle. During each cycle, the aECG segment is first preprocessed followed by the application of two peak detection algorithms PDA and PDB. PDA detects all peaks and valleys within the acceptable range of widths and prominence determined in the calibration step with a required separation of 300 samples. PDB detects all peaks and valleys that surpass the lower bounds of the width and prominence only. PDA determines which peak and valley features meet the specifications for the fQRS detected in the calibration step, while PDB attempts to recover fQRS that may have fallen outside those specifications. PTF is applied to PDA with the best period to model the fQRS being TA. The peaks and valleys detected in PDB that are within 0.75TA of a peak or valley in PDA are removed. The remaining peaks and valleys of PDB are inserted into PDA and PTF is recalculated on PDA for the period TA The fQRS in PDA within the last 1-second the aECG segment are outputted and the aECG is shifted by 1-second for the next cycle.

G. Evaluation

Lullaby is evaluated on the ‘clean’ data sets of the Physionet 2013 Challenge Dataset. The chosen records demonstrated a sensitivity score (SE) above 0.5 as calculated by:

$$SE = \frac{TP}{TP + FN} \qquad ( 11 )$$

where TP represents the number of true fQRS detected and FN represents the number of true fQRS that were not detected.

The accuracy of the fQRS detection of Lullaby is evaluated using the F1-score:

$$F 1 = \frac{2 TP}{TP + FN + FP} \qquad ( 12 )$$

where FP are the number of false fQRS that were detected. Additionally, the average computation time for each segment processed during the real-time processing is measured. The computation time is measured from the beginning of the prepossessing step to the determination of the fQRS.

Bland-Altman [2] analysis is used to directly validate the PTF by comparing the average fHR along the entire aECG segment to the average fHR derived by converting all PTFs in processed windows to heart rate and averaging them.

Lullaby was evaluated on a machine with the following specifications: Intel® Core™ i7-6500U CPU @ 2.50 GHz 2 cores 4 logical processors, 16 GB RAM. The MATLAB R2021a software was used to conduct the evaluation.

### II. Results

Lullaby has an average F1-score of 0.815 on clean data sets and a computation time of 17.8 ms for the real-time phase of the algorithm. Compared to ICA, TS, EKF, and their hybrid/modified models [3], Lullaby is approximately 7 times faster than the next fastest algorithm (Table 1). Furthermore, Lullaby had a better average F1-score for clean data sets than EKF and ICA, and a comparable score to TS for all data sets (Table 1).

TABLE 1
Average F1-score and Computation time of Lullaby
and standard fQRS extraction methods.

| | F1-Score | Computation Time (ms) |
|---|---|---|
| Lullaby | 0.815 | 17.8 |
| FastICA | 0.6008 | 1000 |
| RobustICA | 0.5960 | 300 |
| TS | 0.8265 | 128 |
| EKF | 0.5434 | 1000 |

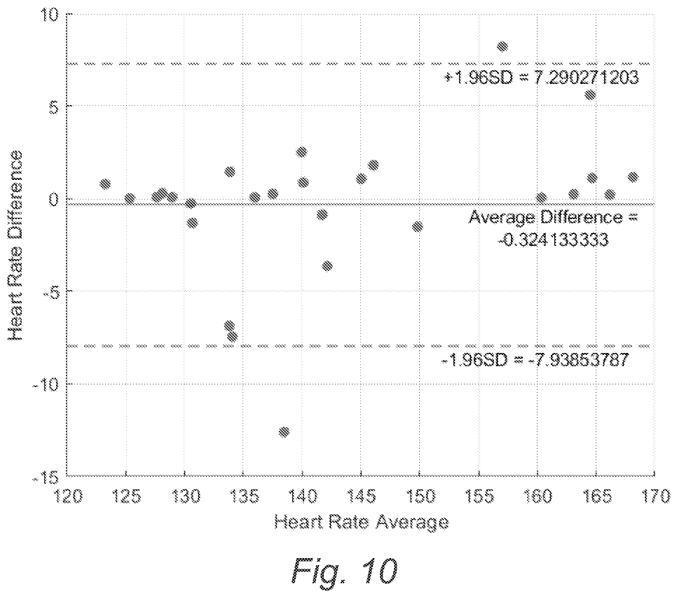

Additionally, the PTF exhibits a fairly accurate representation of the fHR. Bland-Altman analysis (FIG. 10) shows the average PTF deviates from the true heart rate by approximately 8 BPM with 95% confidence and almost zero bias.

### III. Discussion and Conclusions

The ongoing COVID-19 pandemic has accentuated the need for home-based fHR monitoring using a wearable device. However, current wearable devices using aECG are unreliable because they either use fQRS extraction algorithms that cannot run in real-time or too computationally heavy to run directly on the wearable device. The Lullaby algorithm described herein addresses the former concern while the latter was a design consideration but otherwise will be validated in future works. Comparatively, Lullaby is significantly faster than all other comparison algorithms with it being 7 times faster than TS, approximately 17 times faster than RobustICA, approximately 56 times faster than FastICA, and more than 1000 times faster than EKF. In addition, it demonstrated both superior and comparable F1 accuracy to the comparison algorithms.

The novelty of Lullaby stems from the core idea of approximately modelling the fQRS as a periodic occurrence. Using this simple concept, the PTF is able to classify true and false fQRS quickly. Bland-Altman analysis confirms the PTF by itself is able to fairly accurately determine the true fHR within 8 BPM with 95% confidence and no bias. In terms of real-time classification of fQRS in an aECG, PTF demonstrates a strong affinity for the task.

However, for PTF to function the importance of the peak detection steps cannot be discounted. Lullaby heavily uses peak width and prominence features in-order to detect fQRS, which is not a standard method compared to methods such as Pan-Tompkins[4]. The schema is inspired by a similar detection method by Zhang and Yu [5]. Zhang and Yu [5] used the horizontal and vertical distance of peaks as k-means clustering features for classifying noise, mQRS, and fQRS peaks with an F1-score of 0.9547. The width and prominence feature are inherent to peak finding algorithms and can be used specify peak conditions thereby simplifying the Zhang and Yu implementation [5].

Undeniably, Lullaby is at the forefront of real-time fHR computation and demonstrates a high degree of potential for operation on a wearable device. In future works, the Lullaby algorithm will be implemented on a micro-controller to demonstrate direct computation on a wearable device. The accuracy of Lullaby can surely be improved while retaining its real-time capabilities by optimizing standard methods for real-time implementation and improving detection steps. Although in it's infancy, Lullaby marks the first steps in developing a wearable home-based fHR monitoring system that resolves the aforementioned issues of reliability.

Additional details of the invention are found in D. Jilani, T. Le, T. Etchells, M. P. H. Lau, and H. Cao, Lullaby: A novel algorithm to extract fetal QRS in real time using periodic trend feature, IEEE Sensors Lett., vol. 6, no. 9, p. 7003204, 2022; the entire disclosure of which is hereby incorporated by reference in it entirety.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

[1] I. Silva, J. Behar, R. Sameni, T. Zhu, J. Oster, G. D. Clifford, and G. B. Moody, “Noninvasive fetal ecg: the physionet/computing in cardiology challenge 2013,” Computing in Cardiology, vol. 40, pp. 149-152, 2013.
[2] J. M. Bland and D. G. Altman, “Measuring agreement in method comparison studies,” Stat. Methods Med. Res., vol. 8, no. 2, pp. 135-160, June 1999.
[3] S. Sarafan, T. Le, A. M. Naderi, Q.-D. Nguyen, B. T.-Y. Kuo, T. Ghirmai, H.-D. Han, M. P. H. Lau, and H. Cao, “Investigation of methods to extract fetal electrocardiogram from the mother's abdominal signal in practical scenarios,” Technologies, vol. 8, no. 2, 2020. [Online]. Available: https://www.mdpi.com/2227-7080/8/2/33.
[4] J. Pan and W. J. Tompkins, “A real-time qrs detection algorithm,” IEEE Transactions on Biomedical Engineering, vol. BME-32, no. 3, pp. 230-236, 1985.
[5] Y. Zhang and S. Yu, “Single-lead noninvasive fetal ecg extraction by means of combining clustering and principal components analysis,” Medical & Biological Engineering & Computing, vol. 58, no. 2, pp. 419-432, February 2020. [Online]. Available: https://doi.org/10.1007/s11517-019-02087-7.

What is claimed is:

1. A system for detecting fetal peaks in a maternal ECG signal, the system comprising:
an ECG subsystem for collecting a maternal ECG signal that includes fetal peaks; and
a signal processing subsystem configured to:
a) receives the maternal ECG signal;
b) preprocess the maternal ECG signal wherein preprocessing includes removal of mECG R-peaks to form a processed ECG signal that includes both fetal and noise peaks;
c) detect positions of both fetal peaks and noise peaks using a peak detection algorithm;
d) create a plurality of sets of test peak positions wherein each set of test peak positions has its peak positions separated by an associated predetermined time period, the associated predetermined time period for each set being selected from a predetermined range of time period; and
e) align each set of test peak positions with detected positions of both fetal peaks and noise peaks to determine an optimal match with a subset of the detected positions of both fetal and noise peaks, the optimal match identify peaks corresponding to fetal peaks.

2. The system of claim 1, wherein the maternal ECG signal is digitized prior to processing.

3. The system of claim 1, wherein the signal processing subsystem includes a digital signal processor configured to preprocess the maternal ECG signal.

4. The system of claim 1, wherein the signal processing subsystem includes application specific integrated circuits configured to execute steps a) to e).

5. The system of claim 1, wherein the signal processing subsystem includes a computer processor configured to execute steps a) to e).

6. The system of claim 1, wherein the ECG subsystem includes 2 to 4 electrodes for collecting the maternal ECG signal, circuitry for storing and/or processing the maternal ECG signal, and an interface for communicating with a computing device.

7. The system of claim 6, wherein the interface includes an antenna.

8. The system of claim 1, wherein the maternal ECG signal is an abdominal ECG signal.

9. The system of claim 1, wherein positions of both fetal peaks and noise peaks using are stored as a vector of peaks Xₖ of dimension k′ where k′ is the number of peaks and k is an integer label from 1 to k′.

10. The system of claim 9, receiving wherein the optimal match is determined by:
calculating permutations of all peak differences by subtracting elements of Xₖ from its transpose Xₖ^{T} to form a matrix M_{i,j} of dimension k′×k′, elements in the matrix M_{i,j}, corresponding to different sample periods that are used to determine peak positions;
calculating an array of periods denoted an array L_{b} of dimension P×1 where P is the number of time periods and b is a integer label having a value of 1 to P;
constructing a 3-dimensional matrix G_{b,i,j} by dividing M_{i,j} by each element of Lₖ;
determining an optimal M_{i,j} by
determining a temporal error for each element of G_{b,i,j} by rounding each element of G_{b,i,j} to the nearest whole number, subtracting G_{b,i,j} the rounded G_{b,i,j}, and then taking the absolute value of the result as indicated by:
Ĝ_{b,i,j}=|round(G_{b,i,j})−G_{b,i,j}|

summing the elements of Ĝ along its second dimension i to yield Z_{b,j} which represents a total temporal error of the i-th peak (with respect to column of Z) for each period (with respect to row of Z):
finding the row index r and column index c of Z_{b},j with the minimum element gives an optimal period and an optimal reference peak respectively;
selecting the 1st dimension r and 3rd dimension c of G_{k,i,j} as Wᵢ=G_{r,i,c}; and
identifying indexes with W less than a predetermined value as corresponding to true fetal peaks fQRS in Xₖ.

11. The system of claim 10, wherein the array of periods includes each integer in a predetermined range of integers.

12. The system of claim 11, wherein the predetermined range of integers is determined from a range of periods determined from 40 to 200 heart beats per minute.

13. The system of claim 10, wherein preprocessing includes removing baseline wander and signal smoothing.

14. The system of claim 13, wherein baseline wander is removed using a high pass filter or bandpass filter.

15. The system of claim 13, wherein a Savitzky-Golay filter is applied for signal smoothing.

16. The system of claim 13, wherein positions of missing peaks is estimated using a determined periodicity.

17. A computer-implemented method for detecting fetal peaks in a maternal ECG signal comprising:
receiving the maternal ECG signal;
preprocessing the maternal ECG signal wherein preprocessing includes removal of mECG R-peaks to form a processed ECG signal that includes both fetal and noise peaks;
detecting positions of both fetal peaks and noise peaks using a peak detection algorithm;
creating a plurality of sets of test peak positions wherein each set of test peak positions has its peak positions separated by an associated predetermined time period, the associated predetermined time period for each set being selected from a predetermined range of time period; and
aligning each set of test peak positions with detected positions of both fetal peaks and noise peaks to determine an optimal match with a subset of the detected positions of both fetal and noise peaks, the optimal match identify peaks corresponding to fetal peaks.

18. The computer-implemented method of claim 17, wherein the maternal ECG signal is an abdominal ECG signal.

19. The computer-implemented method of claim 17, wherein positions of both fetal peaks and noise peaks using are stored as an array of peaks Xₖ of dimension k′×1 where k′ is the number of peaks.

20. The computer-implemented method of claim 19, receiving wherein the optimal match is determined by:
calculating permutations of all peak differences by subtracting elements of Xₖ from its transpose Xₖ^{T} to form a matrix M_{i,j} of dimension k′×k′, elements in the matrix M_{i,j}, corresponding to different sample periods that are used to determine peak positions;
calculating an array of periods denoted an array L_{b} of dimension P×1 where P is the number of time periods and b is a integer label having a value of 1 to P;
constructing a 3-dimensional matrix G_{b,i,j} by dividing M_{i,j} by each element of L_{b};
determining an optimal M_{i,j} by
determining a temporal error for each element of G_{b,i,j} by rounding each element of G_{b,i,j} to the nearest whole number, subtracting G_{b,i,j} the rounded G_{b,i,j}, and then taking the absolute value of the result as indicated by:
Ĝ_{b,i,j}=|round(G_{b,i,j})−G_{b,i,j}|

summing the elements of Ĝ along its second dimension i to yield Z_{b,j} which represents a total temporal error of the i-th peak (with respect to column of Z) for each period (with respect to row of Z):
finding the row index r and column index c of Z_{b},j with the minimum element gives an optimal period and an optimal reference peak respectively;
selecting the 1st dimension r and 3rd dimension c of G_{b,i,j} as Wᵢ=G_{r,i,c}; and
identifying indexes with W less than a predetermined value as corresponding to true fetal peaks fQRS in Xₖ.

21. The computer-implemented method of claim 20, wherein the array of periods includes each integer in a predetermined range of integers.

22. The computer-implemented method of claim 21, wherein the predetermined range of integers is determined from a range of periods determined from 40 to 200 heart beats per minute.

23. The computer-implemented method of claim 20, wherein preprocessing includes removing baseline wander and signal smoothing.

24. The computer-implemented method of claim 23, wherein baseline wander is removed using a high pass filter or bandpass filter.

25. The computer-implemented method of claim 23, wherein a Savitzky-Golay filter is applied for signal smoothing.

26. The computer-implemented method of claim 23, wherein positions of missing peaks is estimated using a determined periodicity.
